(19) United States
(12) Patent Application Publication
(10) Pub. No.: US 2022/0125696 A1
(43) Pub. Date: Apr. 28, 2022

(54) INFLAMMATION REDUCING COMPOSITION CONTAINING A CANNABIS SATIVA COMPOUND

(71) Applicant: John Christian Haught, Argyle, TX (US)

(72) Inventor: John Christian Haught, Argyle, TX (US)

(21) Appl. No.: 17/490,377

(22) Filed: Sep. 30, 2021

Related U.S. Application Data
(60) Provisional application No. 63/105,319, filed on Oct. 25, 2020.

(51) Int. Cl. A61K 8/34; A61Q 11/00; A61Q 15/00; A61K 8/20; A61K 8/44; A61Q 5/00

(52) U.S. Cl. CPC A61K 8/347; A61Q 11/00; A61Q 5/00; A61K 8/20; A61K 8/44; A61Q 15/00

(57) ABSTRACT

A variety of personal care compositions contain one or more of a cannabinoid, a Lewis acid salt inside of a vehicle to deliver the cannabinoid to a user. The vehicle that is used depends upon where the cannabinoid is to be delivered. For example, oral care compositions contain orally-acceptable carrier(s), skin care compositions contain vehicles that are compatible with the skin, and so on. Personal care compositions can take many product forms and can be used by humans or animals. Personal care compositions include, but are not limited to, mouthwashes, toothpastes, dentifrices, tooth gels, tooth powders, tablets, rinses, subgingival gels, foams, mousses, chewing gums, lipsticks, sponges, flosses, prophy pastes, petrolatum gels, denture products, deodorants, antiperspirants, pet foods and pet chews.

### PRIORITY

This application claims priority to U.S. Provisional Patent Application Ser. No. 63/105,319, filed on Oct. 25, 2020, and entitled, “Inflammation Reducing Composition Containing a Cannabis sativa Compound,” the disclosure of which is incorporated by reference herein.

### BACKGROUND

Cannabinoid compounds are naturally produced in Cannabis plants of the sativa, indicia, or ruderalis varieties. Cannabinoid compounds are produced in these plants from a precursor. See: Luo, X., Reiter, M. A., d'Espaux, L., Wong, J., Denby, C. M., Lechner, A., Zhang, Y., Grzybowski, A. T., Harth, S., Lin, W., Complete biosynthesis of cannabinoids and their unnatural analogues in yeast, Nature 2019, 567, 123-126.

Complete biosynthesis of cannabinoids and their unnatural analogues in yeast. Nature 2019, 567, 123-126. The prenyl group from geranyl pyrophosphate is added to olivetolic acid via an aromatic prenyltransferase to create cannabigerolic acid (CBGA), which is the main precursor of all cannabinoid compounds. The CBGA will then be subsequent converted to either tetrahydrocannabinolic acid (THCA) or cannabidiolic acid (CBDA). Strain specificity of the Cannabis sativa or indica or ruderalis will determine which cannabinoid is present in a higher amount, THCA or CBDA, and the level of each will be governed by the specific synthase enzyme that converts CBGA to either THCA or CBDA. In other words, if THCA synthase is the dominant synthase over CBDA synthase, then that specific strain of Cannabis will produce more of the THCA relative to the CBDA. Conversely, if the CBDA synthase is the dominant synthase, then CBDA will be produced in larger quantities relative to THCA. The subsequent conversion of THCA and CBDA to their neutral forms is a non-enzymatic reaction to remove the carboxylic acid. The neutral forms of the cannabinoids have been shown to be susceptible to light, oxygen and heat degradation to form cannabinol, which has not been shown to have biological activity. See: J W Fairbairn, J A Liebmann, M G Rowan, The stability of cannabis and its preparations on storage, Journal of Pharmacy and Pharmacology, Volume 28, Issue 1, January 1976, Pages 1-7. The harvesting of either tetrahydrocannabinol (THC), cannabidiol (CBD), cannabigerol (CBG), cannabigerolic acid (CBGA), tetrahydrocannabinolic acid (THCA), or cannabidiolic acid (CBDA) is reported in several patents and applications.

Compositions for the oral cavity have been disclosed in the art. For example, U.S. Pat. No. 10,172,786B2, issued on Jan. 8, 2019, to Axim Biotechnologies, disclosed an oral composition of a cannabinoid in combination with lactoferrin. This patent did not disclose the need for antioxidant protection of the cannabinoid, nor the need to deliver the cannabinoid to the gingivae of an end user.

Gel toothpastes have been disclosed in the art. U.S. Pat. No. 3,980,767, issued on Sep. 14, 1976, to Beecham Group Limited, disclosed clear gel compositions in combination with an abrasive tooth composition. The gel compositions have the disadvantage of anionic surfactants, which reduce the delivery of the oil phase to the oral mucosa, specifically the gingivae and buccal regions of the mouth.

Stannous chloride in toothpastes has been disclosed in the art. EP 2246031B1, issued on Jun. 28, 2017 to the Procter & Gamble Company, disclosed a particulate stannous chloride and a silica where the stannous chloride was used as an anti-caking agent in the process of toothpaste making by first mixing together the stannous chloride and silica and then adding the resulting mixture into the toothpaste composition.

International patent publication, WO 1997015277A, filed by Warner Lambert and published on May 1, 1997, disclosed the use of an antimicrobial essential oil of thymol or eucalyptol or methyl salicylate or menthol, a stannous ion, of which stannous chloride was one of those possibilities in an orally acceptable vehicle.

The use of polyamine polymers was disclosed in U.S. Pat. No. 6,162,448, issued on Dec. 19, 2000, to L'Oreal, disclosed the stabilization of retinol in beauty care products. U.S. Pat. No. 6,162,448 does not disclose cannabinoids or other phytochemicals and was not applicable to oral care products.

U.S. Pat. No. 9,902,737, issued on Feb. 27, 2018, to Senomyx Inc., Lewis Acids, such as stannic chloride, were disclosed as electron-pair acceptors in the reaction pathway to create a sweet flavor modifier, which could be used in a toothpaste. This patent does not disclose Cannabis sativa compounds nor the combination of any Michael acceptor with any such compounds, nor Lewis acids with Michael acceptors.

In U.S. Pat. No. 8,653,258, issued on Feb. 18, 2014, to Georgia State University Research Foundation, Inc., the Michael acceptors cinnamaldehyde and (5Z)-4-bromo-5-(bromomethylene)-3-butyl-2(5H)-furanone were disclosed to inhibit quorum sensing pathways and potentially of use in toothpastes and mouthwashes. This patent does not disclose Cannabis sativa compounds nor the combination of any Michael acceptor with any such compounds, nor Lewis acids with Michael acceptors.

In US patent application publication, US 20160158136, filed on Feb. 17, 2016, by the Procter & Gamble Company, Michael acceptors were disclosed as antagonists to TRPV1 and TRPA1 receptors for use in personal care products, cosmetics products, and oral care products. The disclosure also disclosed the use of the Michael acceptor to shift a darker shade to a lighter shade in a personal care composition. Stannous chloride was disclosed as a potential stannous salt in the oral care composition. This patent application did not disclose the use of Cannabis sativa compounds, as its disclosure was targeted to odor reduction and inhibiting the sensory activation of TRPV1 and TRPA1 receptors.

While a variety of personal care compositions have been made and used, it is believed that no one prior to the inventor has made or used any combinations as described herein.

### SUMMARY

It has now surprisingly been found that a combination of cannabinoids with one or more higher reacting antioxidants, for example antioxidants selected from stannous chloride, selenomethionine, selenocysteine, amines/polyamines and combinations thereof, may help to maintain the cannabinoid, cannabidiol (CBD) and/or cannabidiolic acid (CBDa) activity during storage. It has further been surprisingly found that higher reacting antioxidants additionally provide a delivery mechanism for the soft tissues of the mouth. It is also believed that cannabinoids themselves are antioxidants and as such, it is counter intuitive to add higher reacting antioxidants to preserve an antioxidant. The present disclosure illustrates the need for these ‘sacrificial’ antioxidants, which react at a faster rate than do the cannabinoids. Moreover, it is believed that stannous chloride or amines/polyamines are driven to the soft tissues of the mouth, such as the gingivae and buccal tissues of the cheeks, and the tongue will act as such antioxidant.

Without being limited by theory, it is now believed that higher reacting antioxidants are needed in both the oil phase and water phase of an oral care composition to protect cannabinoid(s) contained therein during storage and/or in-use. Exemplary oil phase antioxidants of use are described as those that are hydrophobic with low water or partial water solubility. Exemplary oil phase antioxidants of use may be selected from butylated hydroxytoluene, butylated hydroxyaniline, carotenoids, tocopherol, tocopherol derivatives, stannous chloride and mixtures thereof. Exemplary water phase antioxidants are described as polar and/or hydrophilic compounds with a high degree of water solubility. Exemplary water phase antioxidants of use may be selected from glutathione, ascorbic acid, selenium, selenium derivatives, stannous chloride and mixtures thereof.

Further without being limited by theory, it is believed that a multi-step toothpaste and/or tooth gel may deliver the additionally highly active form of the cannabinoids to the oral mucosa, teeth, and other soft tissues.

The present disclosure therefore directed to, among other developments, useful personal care compositions, such as those used in oral care to clean, preserve, and protect the hard and soft tissues of the mouth and oral cavity. Cannabinoid(s), when delivered to the soft tissues, may provide a defensive layer against virulence factors associated with microbial laden plaque and biofilms.

The present disclosure further relates to a cannabinoid tooth composition containing a primary, secondary, or tertiary nitrogen containing moiety such as those selected from: amino acid, polyamine compound, polyethyleneimine compound and mixtures thereof.

The present disclosure further relates to reacting Lewis acid salts of compatible non-coordinating anion catalyst precursors with the first components of Formula 1 as set forth herein. Examples of Lewis acidic cations useful as cations of the second component include halide salts of reactive transition metal cations.

The present disclosure further relates to personal care compositions comprising the combination of the Lewis acid salts of one or more of stannous chloride and/or selenium salts with a cannabinoid, wherein the cannabinoid is selected from: tetrahydrocannabinol (THC), cannabidiol (CBD), cannabigerol (CBG), acids of the each of the foregoing cannabinoids and combinations thereof.

The present disclosure further relates to a method of detoxifying the skin or gingivae by reducing the production of virulence factors from either the skin or gingivae after application of the personal or oral care compositions by applying the stabilized cannabinoid compositions disclosed herein to the aforementioned skin or gingivae.

### DETAILED DESCRIPTION

The following description of certain examples should not be used to limit the scope. Other examples, features, aspects, and advantages will become apparent to those skilled in the art from the following description, which is, by way of illustration, one of the best modes contemplated for carrying out the material described herein. As will be realized, the chemistry is capable of other different and obvious aspects, all without departing from the intended application. Accordingly, the drawings and descriptions should be regarded as illustrative in nature and not restrictive.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All references, including patent applications, patent publications and non-patent literature, that are referred to in the present specification are incorporated by reference herein, unless it is expressly indicated that they are not incorporated by reference herein.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from about 1 to about 10 should be construed as supporting a range of from about 2 to about 8, from about 3 to about 7, from about 5 to about 6, from about 1 to about 9, from about 3.6 to about 4.6, from about 3.5 to about 9.9 and so forth.

Every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein.

Water solubility is the degree of water solubility is in relation to ascorbic acid, which has a solubility in water of about 330 g/L at 25 degrees C. A high solubility in water would be at least about 50% of the solubility of ascorbic acid in water at 25 C.

As used herein, the terms “about” or “approximately” mean within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20% or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made. The following description of certain examples should not be used to limit the scope. Other examples, features, aspects, and advantages will become apparent to those skilled in the art from the following description, which is by way of illustration, one of the best modes contemplated for carrying out the described compositions. As will be realized, the described is capable of other different and obvious aspects, all without departing from the intended objective. Accordingly, the descriptions should be regarded as illustrative in nature and not restrictive.

The present disclosure is directed to the discovery that a combination of an antioxidant and a Lewis acid, such as a Lewis acid selected from stannous chloride, selenium salts, Michael acceptors and mixtures thereof, with a Cannabis sativa extract of an individual cannabinoid from the Cannabis sativa plant, will stabilize the cannabinoid in an oral or personal care composition, such that the cannabinoid compound will maintain a higher degree of activity than it would in the absence of the Lewis Acid. The cannabinoid, cannabidiol (CBD) when combined with stannous chloride is shown to maintain a high degree of activity when formulated into a toothpaste.

Non-limiting examples of Michael Acceptors of use may be selected from: delta damascene, dihydrojasmone, ascorbic acid, [3-oxo-L-gulofuranolactone], cis-jasmone[3-methyl-2-(2-pentenyl-2-cyclopentenone], 2,5-dimethyl-4-hydroxy-3(2H)-furanone, 5-ethyl-3-hydroxy-4-methyl-2(5H)-furanone, vanillin [4-hydroxy-3-methoxybenzaldehyde], ethyl vanillin, anisaldehyde[4-methoxybenzaldehyde], 3,4-methylenedioxybenzaldehyde, 3,4-dimethoxybenzaldehyde, 4-hydroxybenzaldehyde, 2-methoxybenzaldehyde, benzaldehyde, cinnamaldehyde[3-phenyl-2-propenal], hexyl cinnamaldehyde, alpha-methyl cinnamaldehyde, ortho-methoxy cinnamaldehyde, citral, linalool, geraniol, eugenol and mixtures thereof.

Without wishing to be bound by theory, it is believed that unlike other stannous salts, stannous chloride is able to form a stable structure in water without deionizing into the stannous ion and chloride ion. This particular feature is believed to be unique to stannous chloride due to its ability to attract electron donors, even though it is a Lewis acid having a full octet (eighteen valence electrons.

In the Lewis theory of acid-base reactions, bases donate pairs of electrons and acids accept pairs of electrons. A Lewis acid is therefore any substance, such as an H+ ion, that can accept a pair of nonbonding electrons, meaning that a Lewis acid is an electron pair acceptor. It is believed that this property readily compliments the oxidation-reduction model in that its ability to accept electrons allows a Lewis acid to potentially function as higher level antioxidants to protect from free radicals, essentially protecting the cannabinoids from rapid oxidation.

A Lewis acid salt has the following formula I:

M′(Q)ᵣ

wherein: M′ is a metal or metalloid in its highest oxidation state; r represents the number of Q ligands; and Q is selected from: hydride radicals, dialkyamido radicals, alkoxide and aryloxide radicals, hydrocarbyl and substituted hydrocarbyl radicals, halocarbyl and substituted halocarbyl radicals, and hydrocarbyl, and halocarbyl-substituted organometalloid radicals. In some exemplary Lewis acid salts of use, M′ is selected from: boron, manganese, vanadium, iron, copper, stannous, or selenium and Q is selected from: mineral acids, chlorine, fluorine, sulfur, nitrogen, oxygen, phosphorous, or amino acid, such as methionine, and/or cysteine.

Exemplary mineral acids of use may be selected from: hydrochloric acid, nitric acid, phosphoric acid, phosphonic acid, boric acid, and sulfuric acid. Exemplary organic acids of use may be selected from: sulfonic acids, organophosphorus acids, carboxylic acids such as benzoic acids, propionic acids, phthalic acids, butyric acids, acetic acids, amino acids, and other substituted and unsubstituted organic acids.

Exemplary organic acids of use may be selected from: adipic acid, benzene 1,3,5 tricarboxylic acid, chlorosuccinic acid, choline chloride, cis-aconitic acid, citramalic acid, citric acid, cyclobutane 1,1,3,3 tetracarboxylic acid, cyclohexane 1,2,4,5 tetracarboxylic acid, cyclopentane 1,2,3,4 tetracarboxylic acid, diglycolic acid, fumaric acid, glutamic acid, glutaric acid, glyoxylic acid, isocitric acid, ketomalonic acid, lactic acid, maleic acid, malic acid, malonic acid, nitrilotriacetic acid, oxalacetic acid, oxalic acid, phytic acid, p-toluenesulfonic acid, salicylic acid, succinic acid, tartaric acid, tartronic acid, tetrahydrofuran 2,3,4,5 tetracarboxylic acid, tricarballylic acid, versene acids, 3-hydroxyglutaric acid, 2-hydroxypropane 1,3 dicarboxylic acid, glyceric acid, furan 2,5 dicarboxylic acid, 3,4-dihydroxyfuran-2,5 dicarboxylic acid, 3,4-dihydroxytetrahydrofuran-2,5-dicarboxylic acid, 2-oxo-glutaric acid, dl-glyceric acid, and 2,5 furandicarboxylic acid.

In some exemplary compositions, the proton donor includes a hydroxy carboxylic acid, and in some exemplary compositions, the hydroxy acid includes two or more carboxylic acid groups. In some exemplary compositions, the hydroxy carboxylic acid includes alpha-hydroxy acids and beta-hydroxy acids. Some exemplary alpha-hydroxy acids of use may have two or more carboxylic acid groups including those selected from: tartaric acid, malic acid, citric acid, and isocitric acid. Some exemplary alpha-hydroxy carboxylic acids of use may be selected from: lactic acid, tartronic acid, and malonic acid. In some exemplary compositions, the proton donor may be selected from: citric acid, lactic acid, malic acid, tartaric acid, salicylic acid, oxalic acid, or mixtures thereof. In some exemplary compositions, the proton donor is citric acid.

Since oral cells may contain one or more of the receptors that a bacterial virulence factor may activate, screening on the individual receptors may require the use of engineered cells, such as reporter cells containing the receptor gene of interest. What may further complicate the use of native oral cells, such as gingival cells, is that the expression and activation of a receptor, such as a toll-like receptor, is specific to the function of the cell. Gingival cells are less likely to respond to bacterial virulence factors, due to their constant contact with microbes in the dental plaque. Thus, it is believed that a need exists to have engineered cells for which a direct response can be measured via a reporter system.

In addition to quantifying the virulence of microbial components and byproducts, there also exists a need for an in vitro screen of the inflammatory potential of organic and inorganic molecules, which would allow for pharmokinetic parameters to be determined.

The terms “virulence reduction,” “inflammation reduction,” “detoxification,” “detoxify,” and “detox” are used interchangeably herein to refer to the inactivation of bacterial virulence factors. Exemplary virulence factors of that may be inactivated by the disclosed compositions may be selected from: lipopolysaccharide or endotoxin from gram negative bacteria; or lipoteichoic acid from gram positive bacteria. The inactivation of a virulence factor is defined herein as a virulence factor losing its ability to stimulate a host immune response. An exemplary stimulation of a host immune response would be the activation of one or more of the Interleukin Receptors, such as IL-1, where lipopolysaccharide may have a level of activation on this receptor. A reduction of the activation of this receptor by 30% or more falls under the definition of virulence reduction and/or detoxification. Further, detoxification may be the removal or neutralization of the virulence factor from the system, such as stannous binding to the cell wall components of bacteria, where those cell wall components may comprise lipopolysaccharide and/or lipoteichoic acid.

Exemplary oral care compositions in accordance with the present disclosure may comprise a detoxifying agent that is selected from at least one of: a Lewis acid with a Cannabinoid. In some exemplary oral care compositions, the at least one Lewis acid is selected from: stannous chloride, a selenium salt, amine, polyamine and mixtures thereof, combined with a Cannabinoid, such as Cannabidiol.

Exemplary methods of making oral care compositions in accordance with the present disclosure may include incorporating one or more cannabinoids to deliver anti-gingivitis and/or antioxidant effects. In these exemplary methods, the one or more cannabinoids are naturally derived or artificially derived. In these exemplary methods, cannabinoids are incorporated in the form of hemp oil or cannabis oil, which may contain impurities in an amount of less than about 85%, less than about 80%, less than about 75%, less than about 70%, less than about 65%, less than about 55%, less than about 50%, less than about 45%, less than about 40%, or less than about 35% by weight.

Pathogenesis of gingivitis may involve both bacteria and host responses. Certain methodologies for measuring the virulence factors (J. C. Haught, Xie S., Circello B., Tansky C., Khambe D., Sun Y., Lin Y., Sreekrishna K., Klukowska M., Huggins T., D. J. White. Lipopolysaccharide and lipoteichoic acid binding by antimicrobials used in oral care formulations. Am J Dent 29 (2016) 328-332) in the dental plaques in vitro, and also measuring effects of virulence factors on gingival tissues in vivo, may allow understanding of what virulence factor types are present in the dental plaques, and how the host responds. Importantly, those measurements provide a detailed assessment on the severity of gingivitis in terms of virulence factors of the microbes in dental plaques and healthy status in the host. In addition, these methods help evaluate the effectiveness of a technology in preventing and treating gingivitis.

In practicing the useful methods, a strip of material may be applied to the desired oral surface by the wearer. The side of the material facing the oral surface is at least the side wherein the composition herein is applied. This oral care composition provides a vehicle for the active as well as tackiness between the oral surfaces and the strip of material, holding the strip of material in place for extended periods of time. The period over which the strip of material is used may be, for example, from about one to about thirty minutes.

Useful methods may be used to determine the cellular impact of organic and inorganic molecules, for example if there is an interaction between the molecule and the targeted receptor. Further, the methods may be used to ascertain if a molecule would cause an irritation or inflammatory response, if the receptor for that response is known. The receptor in question could be used in a reporter system as described herein and the molecular impact of the molecule in question determined. The EC50 value of the molecule in question could be determined on the receptor associated with the biological response, thus reducing or eliminating the need to do animal testing.

The disclosure may further include food, pet, and/or personal care compositions comprising stannous chloride, a selenium salt, amine, polyamine, and a cannabinoid in the form of a gel, cream, lotion, serum, toner, aerosol, foam, cosmetic, drink, vitamin, gum, candy, food, or makeup on or in the body and/or to parts of the body other than the oral cavity.

The term “orally acceptable carrier” as used herein means a suitable vehicle or ingredient, which can be used to form and/or apply the present compositions to the oral cavity in a safe and effective manner. Such vehicle may include materials selected from: fluoride ion sources, antibacterial agents, anticalculus agents, buffers, other abrasive materials, peroxide sources, alkali metal bicarbonate salts, thickening materials, humectants, water, surfactants, titanium dioxide, flavor system, sweetening agents, cooling agents, xylitol, coloring agents, other suitable materials and mixtures thereof.

As described in the US Pharmacopia (US Pharm. 2008; 33(10):HS-22-HS-28), an antioxidant, or a free-radical scavenger, is a molecule capable of decreasing or preventing the oxidation of other molecules. Oxidation reactions transfer electrons from a substance to an oxidizing agent. During this process, some free-radicals are produced, which starts chain reactions that damage animal cells. Antioxidants slow down these chain reactions by removing free-radical intermediates and eventually inhibit other oxidation reactions by being oxidized themselves. Antioxidants often play the role of a reducing agent, e.g., thiols or polyphenols.

Free radicals are atoms or molecules that are missing one of two electrons, thus forming the free-radical molecules that seek to complete their structures. When a molecule or atom is missing one of its electrons, it becomes unstable and will try to take another electron from any other molecule in its immediate environment. If a free-radical acquires an electron from the molecule next to it, then that molecule or atom may become a free-radical. In turn, the next free-radical attacks a molecule next to it, and so on, potentially eliciting damage to the biological substrate.

The term “Michael acceptor”, as used herein, refers to alkenes attached to electron-withdrawing groups such as esters, ketones, nitriles, and nitro containing moieties where the beta carbon is the electrophile. The addition reaction is the addition of a nucleophile to a carbanion or to another nucleophile of an .alpha.,.beta.-unsaturated carbonyl compound. The Michael acceptor may have the dual functionality of chelating stain bodies, thus reducing the surface shade from a darker to a lighter color.

The term “functionalized,” as used herein, refers to the state of a moiety that has one or more functional groups introduced to it by way of one or more functionalization reactions known to a person having ordinary skill in the art. Exemplary functionalization reactions that may occur include those selected from: epoxidation, sulfonation, hydrolysis, amidation, esterification, hydroxylation, dihyroxylation, amination, ammonolysis, acylation, nitration, oxidation, dehydration, elimination, hydration, dehydrogenation, hydrogenation, acetalization, halogenation, dehydrohalogenation, Michael addition, aldol condensation, Canizzaro reaction, Mannich reaction, Clasien condensation, Suzuki coupling, and the like. Particularly, functionalization of a moiety replaces one or more hydrogens in the moiety with one or more non-hydrogen groups, including those selected from: alkyl, alkoxyl, cycloalkyl, alkenyl, cycloalkenyl, alkynyl, aryl groups and combinations thereof. Exemplary cycloalkyl groups may be selected from: cyclopentane, cyclohexane, cycloheptane, and the like. Exemplary alkoxy groups may be selected from: methoxy, ethoxy, n-propoxy, isopropoxy, and the like. Exemplary aryl groups may be selected from: benzenes, naphthalenes (2 rings), anthracenes (3 rings), and the like.

Virulence factors are molecules that initiate an immune response from the host cells. In the mouth, microbes and their byproducts, deliver molecules that initiate an immune response from the cells of the host in the gingivae. Exemplary virulence factors may be selected from: bacterial DNA, lipopolysaccharide from gram negative bacteria, lipoteichoic acid from gram positive bacteria, bacterial phospholipids from the bacterial cell wall, bacterial surface proteins, enzymes, proteases, lipases, deoxyribonucleases (DNases), carbohydrates and combinations thereof. Other virulence factors are host generated. Exemplary host generated virulence factors may be selected from: reactive oxygen species, reactive nitrogen species and combinations thereof. Hydrogen peroxide is an exemplary reactive oxygen species of use. Nitric oxide is an exemplary reactive nitrogen species of use.

The word “cannabinoid” as used herein, means any compound that interacts with a cannabinoid receptor and other cannabinoid mimetics, including, but not limited to, those selected from: certain tetrahydropyran analogs (Δ9-tetrahydrocannabinol, Δ8-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1 hydroxy-6,6-dimythel-9H-dibezo[b,d]pyran-9-ol, (−)-(3 S,4 S)-7-hydroxy-delta-6-tetrahydrocannabinol-1,1-dimethylheptyl, (+)-(3 S,4S)-7-hydroxy-Δ-6-tetrahydrocannabinol, and Δ8-tetrahydrocannabinol-11-oic acid); certain piperidine analogs (e.g., (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)); certain aminoalkylindole analogs (e.g., (R)-(+)-[2,3-dihydro-5-methyl-3-(4-morpholinylm-ethyl)-pyrrolo[1,2,3,-de]-1,4-benzoxazin-6-yl]-1-naphthelenyl-methanone); certain open pyran-ring analogs (e.g., 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3′,4′,5′,6′-hexahydrobiphenyl), their salts, solvates, metabolites, and metabolic precursors.

As used herein, the word “cannabidiol” refers to cannabidiol and cannabidiol derivatives. As used in this application, cannabidiol is obtained from industrial hemp extract with a trace amount of THC and/or from cannabis extract using high-CBD cannabis cultivars.

The word “cannabigerol” refers to cannabigerol and cannabigerol derivatives. As used in this application, cannabigerol is derived from industrial hemp extract with a trace amount of THC and/or from cannabis extract.

When cannabinoids are provided as hemp oil and/or cannabis oil, the hemp oil and/or cannabis oil may contain up to 85% impurities, including fatty acids and other plant impurities. The extracted oil is then distilled to increase the cannabinoid concentration. Impurities in hemp oil and cannabis oil may be fatty acids such as linoleic acid and α-linoleic acid, which are natural components of hemp oil or cannabis oil, β-caryophyllene, myrcene, and β-sitosterol.

“Gingivitis” as used herein is consistent with the definition per the FDA monograph (12 CFR Part 356, Vol. 68, No. 103 (2003)) as “[a]n inflammatory lesion of the gingiva that is most frequently caused by dental plaque. Gingivitis is characterized by tissue swelling and redness, loss of stippling (a normal state in which the surface of healthy gingiva is comprised of small lobes), glossy surface, and increased tissue temperature. The gingiva also may bleed upon gentle provocation, such as tooth brushing or may bleed spontaneously. Gingivitis is usually not painful.” Within the monograph, plaque is defined as being composed of multiple bacterial species. Those species exert a constant inflammatory pressure on the host tissues.

When the inflammation progresses to the state of gingivitis, there exists a need to quantify how severe it is and how effective treatments from oral hygiene products are in reducing the inflammatory response. The reduction in inflammatory response due to activation of membrane bound receptors across the gingival cells is termed “detoxification” and measuring the level of detoxification is needed to educate consumers on the efficacy of their oral hygiene, which is lacking in the art.

The term “comprising” as used herein means that steps and ingredients other than those specifically mentioned can be added. This term encompasses the terms “consisting of” and “consisting essentially of” The compositions of the present disclosure can comprise, consist of, and consist essentially of the essential elements and limitations of the disclosure described herein, as well as any of the additional or optional ingredients, components, steps, or limitations described herein.

As used herein, the word “include,” and its variants, are intended to be non-limiting, such that recitation of items in a list is not to the exclusion of other like items that may also be useful in the materials, compositions, devices, and methods of this disclosure.

As used herein, the word “or” when used as a connector of two or more elements is meant to include the elements individually and in combination; for example X or Y, means X or Y or both.

The term “effective amount” as used herein means an amount of a compound or composition sufficient to induce a positive benefit, an oral health benefit, and/or an amount low enough to avoid serious side effects, i.e., to provide a reasonable benefit to risk ratio, within the sound judgment of a skilled artisan.

The terms “oral composition” or “oral care composition” are used interchangeably herein to mean a product that in the ordinary course of usage is retained in the oral cavity for a time sufficient to contact some or all of the dental surfaces and/or oral tissues for purposes of oral activity. The oral composition of the present disclosure may be in various product forms including toothpaste, dentifrice, tooth gel, tooth powders, tablets, rinse, subgingival gel, foam, mouse, chewing gum, lipstick, sponge, floss, prophy paste, petrolatum gel, or denture product. The oral composition may also be incorporated onto strips or films for direct application or attachment to oral surfaces or incorporated into floss.

The term “teeth” as used herein refers to natural teeth as well as artificial teeth or dental prosthesis.

Herein, the terms “tartar” and “calculus” are used interchangeably and refer to mineralized dental plaque biofilms.

The term “polymer” as used herein shall include materials whether made by polymerization of one type of monomer or made by two (i.e., copolymers) or more types of monomers.

The term “water soluble” as used herein means that the material is soluble in water in the present composition. In general, the material should be soluble at 25° C. at a concentration of about 0.1% by weight of the water solvent, at a concentration of about 1%, at a concentration of about 5%, or at a concentration of about 15%.

The term “phase” as used herein means a mechanically separate, homogeneous part of a heterogeneous system.

The term “substantially non-hydrated” as used herein means that the material has a low number of surface hydroxyl groups or is substantially free of surface hydroxyl groups. It may also mean that the material contains less than about 5% total water (free or/and bound).

The term “majority” as used herein means the greater number or part; a number more than half the total.

The term “dispenser” as used herein, means any pump, tube, or container suitable for dispensing compositions such as oral care compositions.

The term “median” as used herein means the middle value in a distribution, above and below which lie an equal number of values.

All percentages, parts and ratios are based upon the total weight of the compositions of the present disclosure, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All measurements referred to herein are made at 25° C. unless otherwise specified.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, skin products, makeup, cosmetics, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs, such as Vicks Vaporub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats.

The present disclosure is also directed towards “oral health compositions” as used herein refers to compositions in a form that is deliverable to a mammal in need thereof via the oral cavity, mouth, throat, nasal passage or combinations thereof. Exemplary oral health care compositions may be selected from: liquid compositions, cough syrups, respiratory preparations, beverages, supplemental waters, pills, soft gels, tablets, capsules, gel compositions, foam compositions, saline washes and combinations thereof. Liquid compositions, gel compositions can be in a form that is directly deliverable to the mouth and throat. These compositions and/or preparations can be delivered by a delivery device selected from: droppers, pump, sprayers, liquid dropper, saline wash delivered via nasal passageway, cup, bottle, liquid filled gel, liquid filled gummy, center filled gum, chews, films, center filled lozenge, gum filled lozenge, pressurized sprayers, atomizers, air inhalation devices, liquid filled compressed tablet, liquid filled gelatin capsule, liquid filled capsule, squeezable sachets, power shots, and other packaging and equipment, and combinations thereof. The sprayer, atomizer, and air inhalation devices can be associated with a battery or electric power source.

The present disclosure is also directed towards a respiratory preparation. Exemplary respiratory preparations may comprise a film forming agent; and a thickening agent. Exemplary respiratory preparations may provide on demand relief. The preparations can work to physically coat the mouth and throat creating a soothing barrier over the epithelial cells that line the throat layer. The preparations can additionally, reduce inflammation and relieve minor pain associated with a cough and/or sore throat. Some exemplary respiratory preparations do not contain a pharmaceutical active.

The present disclosure is also directed to lotion compositions and to absorbent articles, particularly disposable absorbent articles, having a lotion treatment composition applied thereon. Disposable absorbent articles can be baby diapers or feminine hygiene articles, including incontinence devices and catamenial products, such as tampons, sanitary napkins, pantiliners, interlabial products, and the like. For convenience, the disclosure is set forth below with respect to the aspects of a catamenial device, such as a sanitary napkin or pantiliner.

Exemplary cannabinoid absorbent articles can comprise any known or otherwise effective topsheet, such as one which is compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. The topsheet, while capable of allowing rapid transfer of fluid through it, also provides for the transfer or migration of the lotion composition onto an external or internal portion of a body of the wearer. A suitable topsheet can be made of various materials, such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making catamenial products such as sanitary napkins, pantiliners, incontinence pads, and the like, all with the combination of the disclosure.

A cannabinoid lotion composition of the present disclosure comprises at least one cannabinoid and a rheology modifying structuring agent. The lotion composition can further comprise other optional ingredients, like surface energy modifiers. Some exemplary lotion compositions consist essentially of, or consist of, a rheology structurant. Exemplary rheology structurants of use may be selected from: microcrystalline wax, alkyl dimethicone, ethylene glycol dibehenate, ethylene glycol distearate, glycerol tribehenate, glycerol tristearate, ethylene bisoleamide and mixtures thereof. Exemplary lotion compositions can contain a single rheology structurant or a mixture of two or more rheology structurants. In one exemplary lotion composition, the lotion consists of an emulsifier which enable oil to be stabilized in water, O/W, or the emulsifier may be of the type to stabilize water in oil, W/O.

In preparing a cannabinoid lotioned catamenial device according to the present disclosure, the lotion composition can be applied to the outer surface of the absorbent article, such as, for example, the outer surface of the topsheet. Any of a variety of application methods that distribute lubricious materials having a molten or liquid consistency can be used, such as, for example, as set forth in U.S. Pat. No. 5,968,025 and U.S. Pub. App. No. 2005/0208113. Exemplary method may be selected from: spraying, printing (e.g., flexographic printing), coating (e.g., gravure coating), extrusion, dipping, or combinations of these application techniques, e.g., spraying the lotion composition on a rotating surface, such as a calender roll, that then transfers the composition to the outer surface of the sanitary napkin topsheet. Additionally, the manner of applying the lotion composition to a portion of a catamenial device can be such that the substrate or component does not become saturated with the lotion composition. The lotion composition can be applied to the catamenial device at any point during assembly. For example, the lotion composition can also be applied to the outer surface of the topsheet before it is combined with the other raw materials to form a finished catamenial device.

Obtaining a sample of oral matter may be undertaken as follows. The oral matter can include gum-line plaque, subgingival plaque, supragingival plaque, interstitial plaque, gingival crevicular fluid (GCF), gingival biopsy, saliva, or tongue swab. The oral matter may be obtained by any method known in the art, for example, subgingival plaque sample may be collected physically by scraping or by using paper points. The plaque may be collected off of the tooth beneath gums from the sulcus, the developed periodontal pocket, or at the gum line. For example, each paper point can be placed in the pocket between the tooth and the gingiva for 10 seconds. After 10 seconds, a paper point can be removed and placed into a pre-labeled 1.5 ml tube with about 700 μl phosphate-buffered saline. The sampling procedure can be repeated with three more paper points. After all four paper points are collected, the 1.5 ml tube will be closed, vortexed for 30 seconds and placed on dry ice until the samples are stored in a −80° C. freezer. Other methods of collection could include a mechanical device to help release the plaque from the tooth surface, such as a sonic descaler. The oral matter may be obtained both before and after treatment of an oral site from which the oral matter is obtained. An oral site from which the oral matter is obtained includes host tissues and bacterial matters. Further treatment of an oral site may be more than once and may include multiple different treatments, for example a regimen, such as brushing teeth followed by a mouth rinse. In addition to obtaining oral matter before and after completed treatments, oral matter may be obtained between separate treatments, for example between the brushing of teeth followed by the use of mouth rinse.

### Inflammatory Testing Receptor Systems

The present disclosure may involve the reduction of a variety of receptors responsive to bacterial virulence factors. Exemplary receptors that may be reduced are selected from: interleukins, toll-like receptors, tumor necrosis factors, interferons, Class I cytokine receptors (i.e., hematopoietin receptors), Class II cytokine receptors, TNF receptors, immunoglobulin superfamily receptors, nerve growth factors, chemokine receptors and combinations thereof. Exemplary bacterial virulence factors may be selected from the Interleukins (IL1 through IL-17), Toll-Like (TLR), Tumor Necrosis Factor (TNF-alpha), Interferon gamma (IFNGR) and combinations thereof. Exemplary Class I cytokine receptors may be selected from: IL-2, IL-7, IL-9, IL-11, IL-12, IL-13, IL-15, GMCSF, GCSF, OSM and combinations thereof. Exemplary Class II cytokine receptors may be selected from: IFN-alpha, IFN-beta, IFN-gamma, IL-10 and combinations thereof. Exemplary immunoglobulin superfamily receptors may be selected from: TNF-alpha, TNF-beta, CD40, Nerve Growth Factor (NGF), FAS and combinations thereof. Chemokine receptors may be selected from: IL-1, M-CSF, C-Kit and combinations thereof.

The level of receptor activation can be determined by any method known in the art for the type of reporter gene used. For example, if an NFkB-SEAP reporter gene is used, one could measure the production of SEAP in the culture medium. The reporter cells can be treated with virulence factors, or dental plaque matters collected before or after treatments. Expression of the reporter gene will be stimulated and SEAP secreted into the medium when stimulated by virulence factors. The level of reporter gene product SEAP can be readily measured with commercial kits, and will be proportional to the amount of particular types of virulence factors. Similarly, if an NFkB-luciferase, NFkB-beta-lactamase, or other reporter genes are used, available kits can be used to measure the reporter gene products.

A reporter cell refers to a eukaryotic cell, such as, but not limited to, HEK 293T, human monocyte (THP1), Chinese hamster ovary (CHO) cell, murine cells, or monkey kidney epithelial (Vero) cells engineered to express a predetermined number of TLR receptors, for example a single TLR receptor; which is in contrast to gingival cells that express multiple functional TLR receptors. Thus, one type of engineered reporter cells responds to only one type of virulence factors in the dental plaques. In contrast, gingival cells express several types of functional TLRs, and can't be used to identify a single type of virulence factor in dental plaques. The output from gingival cells is the sum of various virulence factors in the dental plaques. HEK 293T cells can be used as reporter cells, as they are easy to maintain and have similar gene expression profiles to oral epithelial cells, making them a closer match to the gene expression of gingival cells, such that the results will mirror in vivo results. In contrast to naturally occurring gingival cells, the reporter cells of the present disclosure are easy to maintain in the lab, and stable in phenotypes. Further, the reporter cells make detection of virulence factors simpler, are more reproducible, increase accuracy, provide higher throughput are more specific and more quantifiable.

The receptor genes are attached to the regulatory sequence of another gene (reporter gene), such as a fluorescent or luminescent gene, which may encode green fluorescent protein, luciferase, alkaline phosphatase, and/or red fluorescent protein. An example of a reporter gene is the SEAP reporter gene. The SEAP reporter gene encodes a secreted enzyme, called embryonic alkaline phosphatase or SEAP. For instance, in certain aspects, the SEAP reporter gene is placed under the control of an interferon-0 minimal promoter fused to five NFkB and AP-1-binding sites. A recombinant Toll-like receptor binds its ligand, and initiates a chain of responses, leading to recruitment of NFkB and API transcription factors to the reporter gene promoter, which induce expression of SEAP. As compared to measuring an immune response the reporter genes of the present disclosure allow rapid, specific and reproducible measurements of virulence factors. Other receptors can be used in combination with Toll-like receptors, for example, nucleotide-binding oligomerization domain (NOD)1 and NOD2 recognize the peptidoglycan derivatives, meso-diaminopimelic acid (meso-DAP) and muramyl dipeptide (MDP), and trigger host innate immune responses.

### Anti-Viral Targets

The antiviral efficacy of the cannabinoid compositions of the present disclosure may be targeted to destroy pathogenic viruses. Pathogenic viruses can be classified into two general types with respect to the viral structure: enveloped viruses and non-enveloped viruses. Some well-known enveloped viruses include herpes virus, influenza virus; paramyxovirus, respiratory syncytial virus, corona virus, HIV, hepatitis B virus, hepatitis C virus, SARS-CoV, and toga virus. Non-enveloped viruses, sometimes referred to as “naked” viruses, include the families Picornaviridae, Reoviridae, Caliciviridae, Adenoviridae and Parvoviridae. Members of these families include rhinovirus, poliovirus, adenovirus, hepatitis A virus, norovirus, papillomavirus, and rotavirus.

Exemplary anti-viral cannabinoid compositions may include a chaotropic agent. Chaotropic agents include agents that disrupt molecular structure, particularly molecular structure formed by nonbonding forces such as hydrogen bonding, Van der Waals interaction, and hydrophobic effect. Chaotropic agents of use may be selected from: urea, thiourea, guanidine-HCl, guanidine thiocyanate, aminoguanidine bicarbonate, guanidine carbonate, guanidine phosphate, aminoguanidine-HCL and mixtures thereof. Although is it known in the art that heat may act as a chaotropic agent, for purposes of this specification, the term chaotropic agent refers to a substance other than heat. This should not be interpreted to exclude the presence of heat from the method of the present disclosure, because as stated hereinbelow, the method of the present disclosure operates over a wide range of temperatures.

In some exemplary cannabinoid compositions, the chaotropic agent is added in an amount of from about 0.25 to about 20 weight percent, based upon the total weight of the antiviral composition. In another aspect, the amount of chaotropic agent is from about 1 to about 15 weight percent, and in yet another aspect, from about 4 to about 12 weight percent, based upon the total weight of the antiviral composition. It will be understood that greater levels of chaotropic agent can be used, if desired, and are expected to perform equally as well.

As described hereinabove, the antiviral composition of this disclosure includes an alcohol, and an enhancer selected from cationic oligomers or polymers, proton donors and chaotropic agents. The composition can further comprise a wide range of optional ingredients, with the proviso that they do not deleteriously affect the antiviral efficacy of the composition. By deleterious is meant that the decrease in the log kill is not de minimus, or in other words, the log kill does not decrease by more than about 0.5. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present disclosure. Exemplary functional classes of ingredients that are of use are described at page 537 of the Handbook. Exemplary ingredients of use includes those of functional classes selected from: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, humectants, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, viscosity increasing agents (aqueous and nonaqueous) and combinations thereof. Examples of other functional classes of materials useful herein that are well known to one of ordinary skill in the art include solubilizing agents, sequestrants, and keratolytics, topical active ingredients, and combinations thereof. Some exemplary antiviral compositions further comprise glycerin.

### Stannous

Stannous ions are used in oral care compositions to deliver benefits such as, for example, enamel care and cavity protection. Suitable stannous sources of use in the present compositions may be selected from: stannous chloride, stannous fluoride, stannous acetate, stannous gluconate, stannous oxalate, stannous sulfate, stannous lactate stannous tartrate and combinations thereof. Some exemplary oral care compositions comprise the stannous salt as the stannous chloride (i.e., SnCl2) and may further comprise stannous chloride dehydrate, stannous chloride anhydrous, and combinations thereof. Other exemplary oral care combinations comprise a combination of stannous salts (e.g., stannous chloride and stannous fluoride) whereby both the desired stannous and fluoride ion are supplied through these salt combinations. Exemplary oral care compositions disclosure may contain stannous ions in the amount ranging from about 0.01% to about 5% (100 to 50,000 ppm), about 0.05% to about 4% (500 to 40,000 ppm), or about 0.075% to about 3% (750 to 30,000 ppm). Some exemplary oral care compositions contain from about 0.1% to about 2% (1,000 to 20,000 ppm), from about 0.5% to about 1.5% (5,000 to 15,000 ppm), or from about 0.2% to about 0.7% (2,000 to 7,000 ppm) stannous ions.

### Metal Ions

The compositions herein may include at least about 0.0001%, by weight of the composition, of metal ions. Some exemplary compositions comprise from about 0.001% to about 5%, alternatively from about 0.001% to about 5%, alternatively from about 0.01% to about 2%, alternatively from about 0.01% to about 2%, by weight of the composition, of metal ions, including stannous ions and optionally include zinc ions.

Exemplary compositions may optionally comprise from about 0.001% to about 4%, by weight of the composition, of zinc ions. Some exemplary compositions comprise metal ions that include at least about 0.005%, by weight of the composition, of zinc ions. In one exemplary composition the composition includes from about 0.005% to about 1%, by weight of the composition, of zinc ions, alternatively from about 0.005% to about 1%, by weight of the composition, of zinc ions. The source of such zinc ions may be any zinc salt, including for example, zinc salts selected from: zinc citrate, zinc sulfate, zinc glycinate, sodium zinc citrate, zinc lactate, and mixtures thereof. In one exemplary composition, the source of zinc ions is a zinc salt selected from: zinc citrate, zinc lactate, and mixtures thereof. In one exemplary composition, the zinc ion source is zinc lactate.

The term “zinc ion(s)” as used herein, is defined to mean the zinc that is in an oral care composition or other oral product and supplied by a source such as zinc salts including zinc lactate. It may refer to the zinc ions that are provided by a zinc source other than zinc salts, added for stabilization purposes.

### Botanicals

Some exemplary oral care compositions herein may further comprise at least one botanical or extract thereof selected from: chamomile, cinnamon, citrus, clove, echinacea, eucalyptus, fennel, ginger, green tea, hop, magnolia, nutmeg, peppermint, pomegranate, rosemary, saffron, sage, spearmint, star anise, turmeric, wintergreen, hops, extracts thereof and mixtures thereof. A lengthy list of botanicals that may be useful herein include those found in U.S. Pat. No. 7,736,629 B2 to Kamath, et al., Jun. 15, 2010.

Hops are the female seed cones of a hop species, Humulus lupulus. Hops are used extensively in brewing for many benefits, including an antibacterial effect that favors the activity of brewer's yeast over less desirable microorganisms. Hops can be subjected to CO2 and ethanol extraction procedures, after which the major components are alpha acids (about 50-70%), beta acids (about 20-35%), hop oils (about 3-7%) and resins (about 5-15%).

### Thickening Agents

In preparing exemplary oral care compositions, it is sometimes necessary to add some thickening material to provide a desirable consistency or to stabilize or enhance the performance of the formulation. Other benefits may include desirable active release characteristics upon use, acceptable shelf-life stability (greater than 4 months to 24 months, or longer), acceptable phase stability (greater than 4 months to 24 months, or longer) of the personal and/or oral care composition.

Thickeners may be present in exemplary personal and/or oral care compositions in the range from about 0.01% to about 15%, or from about 0.05% to about 10%, or from about 0.075% to about 7.5%. Some exemplary oral care compositions contain from about 0.1% to about 5%, or from about 0.5% to about 3%, or from about 0.75% to about 2%, thickeners.

In certain exemplary compositions, the thickening agents are selected from: carboxyvinyl polymers, carrageenan, xanthan gum, hydroxyethyl cellulose and water soluble salts of cellulose ethers such as sodium carboxymethyl cellulose and sodium carboxymethyl hydroxyethyl cellulose, and mixtures thereof. Useful natural gums may be selected from: karaya, gum arabic, arrageenan, alginates, agar, agarose, fucellan, xanthan gum and mixtures thereof (2) natural seed gums may be selected from: guar gum, locust bean gum, tara gum, tamarind gum, psillium gum and mixtures thereof (3) natural plant exudates may be selected from: acacia, tragacanth, karaya ghatti gums and mixtures thereof and, (4) natural fruit extracts may be selected from low and/or high methoxyl pectins. Silica may also be available as a thickening agent, e.g., synthetic amorphous silica. Colloidal magnesium aluminum silicate or finely divided silica can be used as component of the thickening composition to further improve the composition's texture. Some exemplary compositions may comprise colloidal magnesium aluminum silicate or finely divided silica in an amount of from about 0.1 wt % to about 15 wt %, from about 1 wt % to about 10 wt %, about 2 wt % to about 9 wt %, from about 3 wt % to about 8 wt %. In one exemplary composition, the sodium carboxymethylcellulose is present in the oral care composition in the range from about 0.65% to about 5%, or from about 0.75% to about 4%, or from about 1% to about 3%, or from about 1.25% to about 2%. Other thickeners may include carrageenans. In another exemplary composition, the xanthan gum and/or the locust bean gum is present in the oral care composition in the range from about 0.3% to about 5%, from about 0.5% to about 4%, or from about 0.75% to about 3%.

### pH Buffering Agent

Exemplary oral care compositions as described herein may include an effective amount of a buffering agent or pH trimming agents, as used herein, refer to agents that can be used to adjust the pH of the oral care compositions to the above-identified pH range. Useful buffering agents may be selected from: alkali metal hydroxides, ammonium hydroxide, organic ammonium compounds, carbonates, sesquicarbonates, borates, silicates, phosphates, imidazole, and mixtures thereof.

Compositions may comprise buffering agents selected from: phosphates such as monosodium phosphate (monobasic sodium phosphate), dipotassium phosphate, trisodium phosphate (sodium phosphate tribasic dodecahydrate or TSP), sodium tripolyphosphate, phosphoric acid, sodium benzoate, benzoic acid, sodium hydroxide, potassium hydroxide, imidazole, pyrophosphate salts (sodium and potassium salts), sodium gluconate, lactic acid, sodium lactate, phosphoric acid, anhydrous alkali metal carbonates and/or salts thereof, such as sodium carbonate, sesquicarbonates, bicarbonates such as sodium bicarbonate, silicates, bisulfates, citrates (e.g. citric acid, trisodium citrate dehydrate), and combinations thereof.

Some exemplary compositions comprise about 0.01% to 3%, or from about 0.1% to about 1% of sodium gluconate by weight of the composition, and about 0.001% to about 2% may be used to buffer pH.

The pH of the oral care composition is greater than about 4. Some exemplary oral care compositions may have a pH may be between about 4.5 to 9.5, or from about 5 to 7. Some exemplary oral care compositions have a pH of greater than 6. In a calcium carbonate system, the preference is greater than about 7, alternatively from about 8 to 9, or combinations thereof. The pH is typically measured using a ratio of 1:3 of paste:water. For example, whereby about 1 gram of the oral care composition (e.g., toothpaste) is mixed into about 3 grams of deionized water, and then the pH with an industry accepted pH probe that is calibrated under ambient conditions. The pH is measured by a pH meter with Automatic Temperature Compensating (ATC) probe. The pH meter is capable of reading to about 0.001 pH unit. After each usage the electrode should be washed free from the sample solution with water.

### Water

The term “orally acceptable carrier” as used herein means a liquid or semi-solid vehicle such as a paste or a gel for containing the active ingredients of the present disclosure and delivering them to the oral cavity. Water, in addition to polyols, is commonly used as a carrier material in oral compositions due to its many benefits. For example, water is useful as a processing aid, is neutral to the oral cavity and assists in quick foaming of toothpastes. Water, employed in the preparation of commercial oral compositions should be deionized and free of organic impurities. Water may be added as an ingredient in its own right or it may be present as a carrier in other common raw materials such as, for example, sorbitol and surfactant concentrates. The term “total water content” as used herein means the total amount of water present in the oral care composition, whether added separately or as a solvent or carrier for other raw materials but excluding that which may be present as water of crystallization in certain inorganic salts.

The oral care compositions of the present disclosure comprise at least about 20% of a total water content. Some exemplary oral care compositions comprise from about 40% to about 70% of a total water content. Other exemplary oral care compositions comprise from about 45% to about 65%, alternatively from about 40% to about 70%, alternatively from about 50% to about 70%, alternatively from about 50% to about 60%, alternatively from about 45% to about 55%, alternatively from about 55% to about 65%, alternatively from about 40% to about 60%, alternatively about 55%, alternatively combinations thereof, of a total water content. In some exemplary oral care compositions, the water is USP water. This amount of water includes the free water which is added plus that amount which is introduced with other materials such as with sorbitol or silica or any components of the disclosure, as well as the amount of water needed to balance out the formula to 100%. The Karl Fischer method is a one measure of calculating free water.

### Basic Amino Acids

The basic amino acids which can be used in the compositions and methods of the disclosure include not only naturally occurring basic amino acids, such as arginine, lysine, and histidine, but also any basic amino acids having a carboxyl group and an amino group in the molecule, which are water-soluble and provide an aqueous solution with a pH of about 7 or greater.

Accordingly, useful basic amino acids may be selected from: arginine, lysine, serine, citrullene, ornithine, creatine, histidine, diaminobutanoic acid, diaminoproprionic acid, salts thereof or combinations thereof. In some exemplary compositions, the basic amino acids are selected from arginine, citrullene, and ornithine.

In some exemplary compositions, the basic amino acid is arginine, for example, L-arginine, or a salt thereof.

Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

In certain aspects, the basic amino acid is present in an amount corresponding to about 0.1% to about 15%, e.g., about 0.1 wt % to about 10 wt %, e.g., about 0.1 to about 5 wt %, e.g., about 0.5 wt % to about 3 wt % of the total composition weight, e.g., about 1%, 1.5%, 2%, 3%, 4%, 5%, or 8%, wherein the weight of the basic amino acid is calculated as free form.

### Surfactants

Exemplary compositions may comprise anionic surfactants. Useful anionic surfactants may be selected from: water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH3(CH2)mCH2(OCH2CH2)nOSO3X, wherein m is about 6-16, e.g., 10, n is about 1-6, e.g., 2, 3 or 4, and X is Na or, for example sodium laureth-2 sulfate (CH3(CH2)10CH2(OCH2CH2)2OS3ONa); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate, and mixtures thereof. By “higher alkyl” is meant, e.g., a C6-3o alkyl. Some exemplary compositions comprise anionic surfactant selected from: sodium lauryl sulfate sodium ether lauryl sulfate and combinations thereof. When present, the anionic surfactant is present in an amount which is effective, e.g., >0.01% by weight of the formulation, but not at a concentration which would be irritating to the oral tissue, e.g., 1%, and optimal concentrations depend on the particular formulation and the particular surfactant. In one exemplary compositions, the anionic surfactant is present at from about 0.03% to about 5% by weight, e.g., about 1.5%.

In some exemplary compositions, cationic surfactants useful in the present disclosure can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutyl phenoxyethyl dimethylbenzyl ammonium chloride, coconut alkyl trimethyl ammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

In some exemplary compositions, amphoteric surfactants useful in the present disclosure can be broadly defined as suitable betaine type surfactants are described in U.S. Pat. No. 5,180,577, issued to Polefka et al. on Jan. 19, 1993. Typical alkyldimethyl betaines include decylbetaine or 2-(N-decyl-N, N-dimethylammonium) acetate, coconut betaine or 2-(N-coc-N, N-dimethylammonium), myristyl betaine, palmityl betaine, lauryl betaine, cetyl betaine, cetyl betaine, stearyl betaine, etc. Amidobetains are illustrated by cocoamidoethylbetaine, cocoamidopropyl betaine, lauramidopropyl betaine and the like. In some exemplary compositions, betaines of choice include cocoamidopropyl betaine and, in some exemplary compositions, lauramidopropyl betaine.

Useful surfactants may be selected from: anionic, nonionic, amphoteric, zwitterionic, cationic, betaine surfactants, and mixtures thereof. Exemplary oral care compositions may include a surfactant at a level of from about 0.1% to about 50%, from about 0.025% to about 9%, from about 0.05% to about 5%, from about 0.1% to about 2.5%, from about 0.5% to about 2%, or from about 0.1% to about 1% by weight of the total composition. Non-limiting examples of anionic surfactants may include those described at US 2012/0082630 A1 at paragraphs 32, 33, 34, and 35. Non-limiting examples of zwitterionic or amphoteric surfactants may include those described at US 2012/0082630 A1 at paragraph 36; cationic surfactants may include those described at paragraphs 37 of the reference; and nonionic surfactants may include those described at paragraph 38 of the reference.

### Emulsifier/Structuring Agents

Exemplary emulsifier of the description herein, the compositions do not include an emulsifier, but one could be utilized in compositions where emulsification is needed, such as skincare compositions and cosmetics. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

The topical skincare composition may comprise from about 0.01% to about 15%, from about 0.1% to about 10%, or from about 0.5% to about 5%, by weight of the composition, of a structuring agent. In some exemplary compositions, structuring agents are oil-in-water emulsions. Without being limited by theory, it is believed that the structuring agent may assist in providing rheological characteristics to the composition which contribute to the stability of the composition. For example, the structuring agent tends to assist in the formation of the liquid crystalline gel network structures. The structuring agent may also function as an emulsifier or surfactant.

Nonlimiting examples of structuring agents may include stearic acid, palmitic acid, stearyl alcohol, cetyl alcohol, behenyl alcohol, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. Some exemplary topical skincare compositions may comprise a structuring agent selected from: stearic acid, palmitic acid, stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units and mixtures thereof.

### Thickening Agent

Some exemplary topical skincare compositions may further comprise from about 0.01% to about 10%, from about 0.1% to about 5%, or from about 0.2% to about 5% by weight of the topical skincare composition, of a thickening agent. The thickening agent may be provided in any amount known to one of ordinary skill in the art to facilitate achieving the desired viscosity in combination with the other ingredients in the skin care composition. Thickening agents may be used to adjust the viscosity of a composition without substantially changing its other properties. Thickening agents may also improve the suspension of other ingredients. Some thickening agents may also function as stabilizers when they are used to maintain the stability of an emulsion. Thickening agents may be especially useful in products forms such as ointments.

Non-limiting examples of thickeners that may be suitable for use herein may be selected from: gums, modified gums, starches, modified starches, clays, and cross-linked water swellable polymers. Other non-limiting examples of thickeners are disclosed in U.S. Publication No. 2008/0051497 and U.S. Pat. No. 9,795,552. Exemplary topical skincare compositions may comprise a thickening agent selected from: carboxylic acid polymers, crosslinked polyacrylate polymers, polyacrylamide polymers, polysaccharides, gums, crosslinked vinyl ether/maleic anhydride copolymers, crosslinked poly(N-vinylpyrrolidones), and mixtures thereof. The topical skincare composition may comprise a thickening agent selected from carboxylic acid polymers, crosslinked polyacrylate polymers, polyacrylamide polymers, and mixtures thereof, more selected from crosslinked polyacrylate polymers, polyacrylamide polymers and mixtures thereof.

### Benefit Phase

As noted herein, personal care compositions can include a benefit phase. These exemplary compositions may comprise from about 0.1% to about 50%, by weight of the composition, of a benefit phase. The benefit phase can be hydrophobic and/or anhydrous. The benefit phase can also be substantially free of or free of surfactant. In particular, the benefit phase can comprise from about 0.1% to about 50%, by weight of the rinse-off personal care composition, of a benefit agent. The benefit phase can include, for example, from about 0.5% to about 20%, by weight of the rinse-off personal care composition, of a benefit agent.

A benefit phase can have a particle size of about 4 to about 500 μm, from about 5 to about 300 μm, from about 6 to about 100 μm, or from about 10 to about 50 μm. The particle size is measured in neat product under a differential interference contrast optical microscope with a 10× objective lens. The particle size distribution is counted manually. All benefit phase particles are assumed as uniform spheres in this application. For irregular shaped benefit phase particles, the longest axis is used as the diameter for the particle size distribution counting. The number weighted average of all lipid particles is defined as the average lipid particle size. This measurement can also be accomplished with a computer algorithm.

A benefit phase can have a viscosity as measured by a standard rheometer, such as a Brookfield R/S plus. A sample of 2.5 mL is measured with a spindle C75-1 at a shear rate of 2 s−1 at 25° C. A benefit phase can generally have a viscosity of about 200 cP to about 15,000 cP. However, it has been discovered that lower viscosity benefit phases (i.e. less than about 2000 cP) can be advantageous for manufacturing as it is easier to blend the benefit phase and the surfactant phase. Thus, for example, the benefit phase has a viscosity of about 200 cP to about 1800 cP or from about 300 cP to about 1500 cP.

A benefit agent can include a liquid benefit agent. A liquid benefit agent is considered liquid if that is its natural state at room temperature (i.e. 23° C.). A liquid benefit agent can have a viscosity of less than about 1000 cP, less than about 800 cP, or less than about 600 cP, and can be measured with a standard rheometer.

The liquid benefit agent can have a hydrophobic component. The hydrophobic component can be, for example, a water-dispersible, non-volatile liquid. The water-dispersible, non-volatile liquid benefit agents can have a Vaughn Solubility Parameter (VSP) ranging from about 5 to about 14. Non-limiting examples of hydrophobic benefit materials having VSP values ranging from about 5 to about 14 include the following: Cyclomethicone (5.9), Squalene (6.0), Isopropyl Palmitate (7.8), Isopropyl Myristate (8.0), Castor Oil (8.9), Cholesterol (9.6), Butylene Glycol (13.2), soybean oil, olive oil (7.87), mineral oil (7.1), and combinations thereof.

Non-limiting examples of glycerides suitable for use as liquid benefit agents herein can include castor oil, safflower oil, corn oil, walnut oil, peanut oil, olive oil, cod liver oil, almond oil, avocado oil, palm oil, sesame oil, soybean oil, vegetable oils, sunflower seed oil, coconut oil, cottonseed oil, jojoba oil, and combinations thereof.

Non-limiting examples of glyceride derivatives suitable for use as liquid benefit agents herein can include cationic derivatives, amino acid derivatives, alkanolamide derivatives, esterified derivatives, ether derivatives, hydrogenated derivatives, and combinations thereof. Non-limiting examples of metathesized oligomers suitable for use as liquid benefit agents herein can include oligomers derived from metathesis of unsaturated polyol esters, for example. Exemplary metathesized unsaturated polyol esters and their starting materials are set forth in U.S. Patent Application U.S. 2009/0220443 A1, which is incorporated herein by reference. The unsaturated polyol ester is an unsaturated ester of glycerol. Sources of unsaturated polyol esters of glycerol include synthesized oil, plant oils, algae oils, bacterial derived oils, and animal oils, combinations of theses, and the like. Representative examples of plant oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, tung oil, castor oil, high erucic rape oils, Jatropha oil, combinations of theses, and the like. Representative examples of animal oils include fish oil and the like. A representative example of a synthesized oil includes tall oil, which is a byproduct of wood pulp manufacture.

Other examples of unsaturated polyol esters include diesters such as those derived from ethylene glycol or propylene glycol, esters such as those derived from pentaerythritol or dipentaerythritol, or sugar esters such as SEFOSE®. Non-limiting examples of sucrose polyesters suitable for use include SEFOSE® 1618S, SEFOSE® 1618U, SEFOSE® 1618S B6, SEFOSE® 1618U B6, Sefa Cottonate, Sefa C895, Sefa C1095, SEFOSE® 1618S B4.5, all available from The Procter and Gamble Co. of Cincinnati, Ohio. Other examples of suitable natural polyol esters may include but not be limited to sorbitol esters, maltitol esters, sorbitan esters, maltodextrin derived esters, xylitol esters, and other sugar derived esters. The poloyl ester oligomers may also be modified further by partial hydroformylation of the unsaturated functionality to provide one or more OH groups and an increase in the oligomer hydrophilicity.

Non-limiting examples of hydrocarbons suitable for use as liquid benefit agents herein can include carbon chain length of about C6 or higher including alkanes, polyalkanes, olefins, polyolefins and combinations thereof. Non-limiting examples include mineral oil.

Non-limiting examples of glyceride derivatives for use as liquid benefit agents here in can include cationic derivatives, amino acid derivatives, alkanolamide derivatives, esterified derivatives, ether derivatives, hydrogenated or partially hydrogenated oils and their derivatives, and combination thereof.

Non-limiting examples of alkyl esters suitable for use as liquid benefit agents herein can include isopropyl esters of fatty acids and long chain esters of long chain (i.e. C10-C16) fatty acids, non-limiting examples of which can include isopropyl palmitate, isohexyl palmitate and isopropyl myristate.

Non-limiting examples of silicone oils suitable for use as hydrophobic liquid skin benefit agents herein can include dimethicone copolyol, dimethylpolysiloxane, diethylpolysiloxane, mixed C1-C30 alkyl polysiloxanes, phenyl dimethicone, dimethiconol, and combinations thereof. Nonlimiting examples of silicone oils useful herein are described in U.S. Pat. No. 5,011,681. Still other suitable hydrophobic skin benefit agents can include milk triglycerides (e.g., hydroxylated milk glyceride) and polyol fatty acid polyesters.

The benefit agent may also be non-liquid. Some examples of non-liquid benefit agents include hydrocarbons. Non-limiting examples of hydrocarbons suitable for use as non-liquid benefit agents herein can include petrolatum, microcrystalline wax, polyalkanes, polyolefins, and combinations thereof.

Non-limiting examples of glycerides suitable for use as non-liquid benefit agents herein can include plant waxes, animal fats, hydrogenated or partially hydrogenated plant oils, e.g. shea butter, hydrogenated soybean oil, hydrogenated palm, lanolin, lard, and combinations thereof.

Non-limiting examples of metathesized glycerides suitable for use as non-liquid benefit agents herein can include metathesized palm oil, hydrogenated or partially hydrogenated metathesized soybean oil and canola oil, and combinations thereof.

Non-limiting examples of alkyl esters suitable for use as non-liquid benefit agents herein can include isopropyl esters of fatty acids and long chain esters of long chain (i.e. C10-C24) fatty acids, e.g., cetyl ricinoleate, non-limiting examples of which can include cetyl riconoleate and stearyl riconoleate. Other examples can include hexyl laurate, isohexyl laurate, myristyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as non-liquid benefit agents can include oleyl myristate, oleyl stearate, oleyl oleate, and combinations thereof.

Non-limiting examples of polyglycerin fatty acid esters suitable for use as non-liquid benefit agents herein can include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as non-liquid benefit agents herein can include lanolin, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Non-limiting examples of silicones suitable for use herein can include silicone elastomers.

### Chelants

Exemplary oral care compositions of the present disclosure may comprise one or more chelants, also known as chelating agents. The term “chelant”, as used herein means a bi- or multidentate ligand having at least two groups capable of binding to stannous ions and in some exemplary oral care compositions, other divalent or polyvalent metal ions and which, at least as part of a chelant mixture, are capable of solubilizing the stannous ions and other optional metal ions within the oral care compositions. Groups capable of binding to stannous and other metal ions include carboxyl, hydroxl and amine groups. Typically, those chelants useful herein will also form water soluble stable complexes with the stannous ions.

Suitable chelants herein include C2-C6 dicarboxylic and tricarboxylic acids, such as succinic acid, malic acid, tartaric acid and citric acid; C3-C6 monocarboxylic acids substituted with hydroxyl, such as gluconic acid; picolinic acid; amino acids such as glycine; salts thereof and mixtures thereof. The chelants can also be a polymer or copolymer in which the chelating ligands are on the same or adjacent monomer.

In some exemplary oral care compositions, chelant polymers are polyacids selected from: a homopolymer of a monomer, a co-polymer of two or more different monomers, and a combination thereof, wherein the monomer or at least one of the two or more different monomers is selected from acrylic acid, methacrylic acid, itaconic acid, maleic acid, glutaconic acid, aconitic acid, citraconic acid, mesaconic acid, fumaric acid and tiglic acid.

Some exemplary oral care compositions comprise methylvinylether/maleic acid (PVM/MA) copolymer. Also suitable are polyphosphates, such as tripolyphosphates and/or sodium hexametaphosphate. Longer chain linear polyphosphates, though good chelants, are susceptible to hydrolysis in aqueous compositions. Upon hydrolysis they form Olihophosphates which form insoluble zinc complexes. In one aspect, the composition comprises less than about 0.1% of polyphosphates having a chain length of four or more.

Exemplary oral care compositions may further comprise one or more chelating agents able to complex calcium found in the cell walls of the bacteria. Binding of this calcium weakens the bacterial cell wall and augments bacterial lysis.

Another group of agents suitable for use as chelating or anti-calculus agents in the present disclosure are the soluble pyrophosphates. The pyrophosphate salts used in the present compositions can be any of the alkali metal pyrophosphate salts. In certain aspects, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide least 0.1 wt. % pyrophosphate ions, e.g., about 0.1 to 3 wt. %, e.g., about 0.1 to 2 wt. %, e.g., about 0.1 to 1 wt. %, e.g., about 0.2 to 0.5 wt. %. The pyrophosphates also contribute to preservation of the compositions by lowering water activity.

Some exemplary oral care compositions comprise organic acid chelants selected from: citrate, malate, tartrate, gluconate, succinate, lactate, malonate, maleate, and mixtures thereof, whether added in their free acid or salt forms.

### Polymers

Exemplary oral care compositions may further comprise one or more polymers selected from: polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain aspects include 1:4 to 4:1 copolymers of maleic anhydride or acid with another polymerizable ethylenically unsaturated monomer, for example, methyl vinyl ether (methoxyethylene) having a molecular weight (M.W.) of from about 30,000 to about 1,000,000. These copolymers are available for example as Gantrez AN 139 (M.W. 500,000), AN 1 19 (M.W. 250,000) and S-97 Pharmaceutical Grade (M.W. 70,000), of GAF Chemicals Corporation.

Other operative polymers of use may include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

### Abrasives

Dental abrasives are useful in oral care compositions for their ability to remove surface stains and pellicle and for polishing the teeth. The oral care compositions of the present disclosure may contain a dental abrasive. Dental abrasives useful in the oral care composition of the subject disclosure include many different materials. The material selected must be one which is compatible with the composition of interest and does not excessively abrade dentin. Suitable abrasives include, for example, silicas including gels and precipitates, fused silica, insoluble sodium polymetaphosphate, hydrated alumina, and resinous abrasive materials such as particulate condensation products of urea and formaldehyde, and calcium carbonates, both natural and precipitated.

Natural calcium carbonate is found in rocks such as chalk, limestone, marble and travertine. It is also the principle component of eggshells and the shells of mollusks. The natural calcium carbonate abrasive of the disclosure is typically a finely ground limestone which may optionally be refined or partially refined to remove impurities. For use in the present disclosure, the material has an average particle size of less than 10 microns, e.g., about 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, some exemplary compositions comprise no more than about 0.01%, or no more than about 0.004% by weight of particles would not pass through a 325 mesh. The material has strong crystal structure, and is thus much harder and more abrasive than precipitated calcium carbonate. The tap density for the natural calcium carbonate is for example between about 1 and 1.5 g/cc, e.g., about 1.2 for example about 1.19 g/cc. There are different polymorphs of natural calcium carbonate, e.g., calcite, aragonite and vaterite, calcite being preferred for some exemplary compositions in accordance with the present disclosure. An example of a commercially available product suitable for use in the present disclosure includes Vicron® 25-11 FG from Minerals Technologies INC.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present disclosure, the particles are small, e.g., having an average particle size of about 1-5 microns, and e.g., no more than about 0.1%, no more than about 0.05% by weight of particles which would not pass through a 325 mesh. The particles may for example have a D50 of about 3-6 microns, for example about 3.8-4.9, e.g., about 4.3; a D50 of about 1-4 microns, e.g., about 2.2-2.6 microns, e.g., about 2.4 microns, and a D10 of about 1-2 microns, e.g., about 1.2-1.4, e.g., about 1.3 microns. The particles have relatively high water absorption, e.g., at least 25 g/100 g, e.g. 30-70 g/100 g. Examples of commercially available products suitable for use in the present disclosure include, for example, Carbolag® 15 Plus from Lagos Industria Quimica.

Silica dental abrasives of various types are preferred herein because of their unique benefits of exceptional dental cleaning and polishing performance without unduly abrading tooth enamel or dentine. Silica abrasive polishing materials herein, as well as other abrasives, generally have an average particle size ranging from about 0.1 to 30 μm, and from about 5 to 15 μm. The abrasive can be precipitated silica or silica gels such as the silica xerogels marketed under the trade name “Syloid” by the W.R. Grace & Company, Davison Chemical Division and precipitated silica materials such as those marketed by the J.M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present disclosure are described in more detail in U.S. Pat. Nos. 4,340,583; 5,603,920; 5,589,160; 5,658,553; 5,651,958; and 6,740,311.

Alternatively, mixtures of dental abrasives can be used, such as mixtures of the various grades of Zeodent® silica abrasives as listed above, or mixtures of the silica abrasives and calcium-containing abrasives. Dental solution, mouth spray, mouth wash, and non-abrasive gel compositions of the subject disclosure typically contain little or no abrasive.

The present disclosure in its method aspect involves applying to the oral cavity a safe and effective amount of the compositions described herein.

The compositions and methods according to the disclosure can be incorporated into oral compositions for the care of the mouth and teeth such as toothpastes, transparent pastes, gels, mouth rinses, sprays and chewing gum.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative aspects within the scope of the present disclosure. The examples are given solely for illustration and are not to be construed as limitations of this disclosure as many variations are possible without departing from the spirit and scope thereof. Various modifications of the disclosure in addition to those shown and described herein should be apparent to those skilled in the art and are intended to fall within the appended claims.

### Sweetener

The oral care compositions herein may include a sweetening agent. These include sweeteners such as saccharin, dextrose, sucrose, lactose, xylitol, maltose, levulose, aspartame, sodium cyclamate, D-tryptophan, dihydrochalcones, acesulfame, sucralose, neotame, and mixtures thereof. Sweetening agents are generally used in oral care compositions at levels of from about 0.005% to 5%, alternatively about 0.01% to 1%, by weight of the composition, alternatively from about 0.1% to 0.5%, alternatively combinations thereof.

### Fluoride Ion Source

The oral care active may include an effective amount of an anti-caries agent. In one aspect, the anti-caries agent is a fluoride ion source. The fluoride ion may be present in an amount sufficient to give a fluoride ion concentration in the composition at 25° C., and/or in one aspect can be used at levels of from about 0.0025% to 5% by weight of the composition, alternatively from about 0.005% to 2.0% by weight of the composition, to provide anti-caries effectiveness.

Representative fluoride ion sources include: stannous fluoride, sodium fluoride, potassium fluoride, amine fluoride, ammonium fluoride, sodium monofluorophosphate, sodium fluorosilicate, zinc fluoride, and mixtures thereof. In one aspect the oral care composition contains a fluoride source selected from stannous fluoride, sodium fluoride, and mixtures thereof. In one aspect, the fluoride ion source is sodium monofluorophosphate, and wherein the composition comprises about 0.0025% to 2% of the sodium monofluorophosphate by weight of the composition, alternatively from about 0.5% to 1.5%, alternatively from about 0.6% to 1.7%, alternatively combinations thereof. In another aspect, the composition comprises from about 0.0025% to 2% of a fluoride ion source by weight of the composition. The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference.

In certain aspects the fluoride ion source includes stannous fluoride, sodium fluoride, sodium monofluorophosphate as well as mixtures thereof. Where the formulation comprises calcium salts, the fluoride salts are salts wherein the fluoride is covalently bound to another atom, e.g., as in sodium monofluorophosphate, rather than merely ionically bound, e.g., as in sodium fluoride. In certain aspects, the sole fluoride source is stannous fluoride.

### Anti-Calculus Agent

The oral care compositions may include an effective amount of an anti-calculus agent, which in one aspect may be present from about 0.05% to 50%, alternatively from about 0.75% to 25%, alternatively from about 0.1% to 15%. One example is a pyrophosphate salt as a source of pyrophosphate ion. In one aspect, the composition comprises tetrasodium pyrophosphate (TSPP) or disodium pyrophosphate or combinations thereof, about 0.01% to 2%, more from about 0.1% to 1% of the pyrophosphate salt by weight of the composition. Without wishing to be bound by theory, TSPP may provide not only calcium chelating thereby mitigating plaque formation, but also may also provide the additional benefit of monofluorophosphate stabilization (in those formulations containing monofluorophosphate).

### Humectants

The oral care compositions herein may contain humectants. The humectant serves to keep the oral care composition from hardening upon exposure to air and to reduce evaporation to give a moist feel to the mouth, and, for particular humectants, certain humectants can impart a desirable sweetness of flavor.

Suitable humectants for the present disclosure include edible polyhydric alcohols such as glycerin, sorbitol, xylitol, butylene glycol, polyethylene glycol, propylene glycol, and combinations thereof. In one aspect, the humectant is selected from sorbitol, glycerin, and combinations thereof. In yet another aspect, the humectant is sorbitol. In one aspect, the oral care composition comprises from about 20% to less than 80% of humectants by weight of the composition, from about 30% to 50%. In yet another aspect, the oral care composition contains about 30% to 50% of sorbitol by weight of the oral care composition.

The humectant, on a pure humectant basis, generally includes about 15% to 70% in one aspect or about 30% to 65% in another aspect by weight of the composition.

### Coloring Agents

The oral care compositions herein may include a coloring agent (i.e., pigments, dyes and opacifiers). The coloring agent may be in the form of an aqueous solution, about 1% coloring agent in a solution of water. Titanium dioxide may also be added to the present composition. Titanium dioxide is a white powder which adds opacity to the oral care compositions. Titanium dioxide generally comprises from about 0.25% to about 5%, by weight of the oral care composition.

### Flavoring Agents

The oral care compositions of the disclosure may also include a flavoring agent. Flavoring agents which are used in the practice of the present disclosure include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain aspects employ the oils of peppermint and spearmint.

The oral care compositions herein may include from about 0.001% to about 5%, alternatively from about 0.01% to about 4%, alternatively from about 0.1% to about 3%, alternatively from about 0.5% to about 2%, alternatively combination thereof, of a flavorant composition by weight of the oral care composition.

Examples of flavor compositions or flavor ingredients include: mint oils, wintergreen, clove bud oil, cassia, sage, parsley oil, marjoram, lemon, orange, propenyl guaethol, heliotropine, 4-cis-heptenal, diacetyl, methyl-p-tert-butyl phenyl acetate, methyl salicylate, ethyl salicylate, 1-menthyl acetate, oxanone, a-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, a-terpineol, linalool, limonene, citral, neral, geranial, geraniol nerol, maltol, ethyl maltol, anethole, dihydroanethole, carvone, menthone, beta-damascenone, ionone, gamma-decalactone, gamma-nonalactone, y-undecalactone, or combinations thereof. Generally suitable flavor ingredients are chemicals with structural features and functional groups that are less prone to redox reactions. These include derivatives of flavor ingredients that are saturated or contain stable aromatic rings or ester groups.

### Sensorial Agents

Sensates such as cooling, warming, and tingling agents are useful to deliver signals to the consumer. The most well-known cooling agent is menthol, particularly 1-menthol, which is found naturally in peppermint oil. Among synthetic cooling agents, many are derivatives of or are structurally related to menthol, i.e., containing the cyclohexane moiety, and derivatized with functional groups including carboxamide, ketal, ester, ether and alcohol. Examples include the p-menthanecarboxamide compounds such as N-ethyl-p-menthan-3-carboxamide (known commercially as “WS-3”). An example of a synthetic carboxamide cooling agent that is structurally unrelated to menthol is N,2,3-trimethyl-2-isopropylbutanamide. Additional exemplary synthetic cooling agents include alcohol derivatives such as 3-1-menthoxy-propane-1,2-diol, isopulegol, p-menthane-3,8-diol; menthone glycerine acetal (known commercially as “MGA”); menthyl esters such as menthyl acetate, menthyl acetoacetate, menthyl lactate, and monomenthyl succinate.

Additional agents that are structurally unrelated to menthol but have been reported to have a similar physiological cooling effect include alpha-keto enamine derivatives described in U.S. Pat. No. 6,592,884, including 3-methyl-2-(1-pyrrolidinyl)-2-cyclopenten-1-one (3-MPC), 5-methyl-2-(1-pyrrolidinyl)-2-cyclopenten-1-one (5-MPC); 2,5-dimethyl-4-(1-pyrrolidinyl)-3 (2H)-furanone (DMPF); icilin (also known as AG-3-5, chemical name 142-hydroxyphenyl]-4-[2-nitrophenyl]-1,2,3,6-tetrahydropyrimidine-2-one).

Some examples of warming agents include ethanol; nicotinate esters, such as benzyl nicotinate; polyhydric alcohols; nonanoyl vanillyl amide; nonanoic acid vanillyl ether; vanillyl alcohol alkyl ether derivatives such as vanillyl ethyl ether, vanillyl butyl ether, vanillyl pentyl ether, and vanillyl hexyl ether; isovanillyl alcohol alkyl ethers; ethylvanillyl alcohol alkyl ethers; veratryl alcohol derivatives; substituted benzyl alcohol derivatives; substituted benzyl alcohol alkyl ethers; vanillin propylene glycol acetal; ethylvanillin propylene glycol acetal; ginger extract; ginger oil; gingerol; zingerone; or combinations thereof.

Examples of some tingling agents include capsaicin; homocapsaicin, jambu oleoresin, zanthoxylum peperitum, saanshool-I, saanshool II, sanshoamide, piperine, piperidine, spilanthol, 4-(1-methoxymethyl)-2-phenyl-1,3-dioxolane, or combinations thereof.

The oral care compositions herein can further include herbal ingredients such as extracts of chamomile, oak bark, rosemary and salvia. These, and some of the herb-derived flavoring components can be included at levels just sufficient to provide a contribution to the flavor or they can be added at higher levels, such as 1% or more, in order to provide a greater therapeutic effect.

Other suitable flavorant components are described in Fenaroli's Handbook of Flavor Ingredients, Third Edition, Volumes 1 & 2, CRC Press, Inc. (1995), and Steffen Arctander's Perfume and Flavour Chemicals, Volumes 1 & 2 (1969).

### Other Ingredients

Exemplary oral care compositions may comprise the usual and conventional ancillary components such as anti-microbial agents, fluoride ions, and other ingredients that are known to one skilled in the art. It will be appreciated that selected components for the oral care compositions must be chemically and physically compatible with one another.

### Method of Use

The present disclosure also relates to methods for treating the oral cavity comprising administering to the oral care cavity an oral care composition according to the present disclosure. The term “treating” refers to cleaning and polishing teeth. The method of use herein comprises contacting a subject's dental enamel surfaces and oral mucosa with the oral care compositions according to the present disclosure. The method of treatment may be by brushing with a toothpaste or rinsing with a toothpaste slurry or mouthrinse. Other methods include contacting the topical oral gel, mouthspray, toothpaste, dentifrice, tooth gel, tooth powders, tablets, subgingival gel, foam, mouse, chewing gum, lipstick, sponge, floss, petrolatum gel, or denture product or other form with the subject's teeth and oral mucosa. Depending on the application, the oral care composition may be used as frequently as toothpaste, or may be used less often, for example, weekly, or used by a professional in the form of a prophy paste or other intensive treatment.

The present disclosure also relates to a method of treating gingivitis and plaque with reduced inflammation, by using the present compositions. Additionally, provided are methods of oral care compositions, which have caries, gingivitis, plaque, tartar, stain, sensitivity, aesthetics, breath, mouthfeel, and cleaning benefits. The benefits of these compositions may increase over time when the composition is repeatedly used.

### Examples and Data

Exemplary oral compositions A through D are set forth in Table 1, as is known in the art.

TABLE 1

| Ingredient | A | B | C | D |
|---|---|---|---|---|
| Sodium Fluoride | 0.243% | 0.243% | 0% | 0% |
| Stannous Fluoride | 0% | 0% | 0.454% | 0.454% |
| Sodium Saccharin | 0.400% | 0.400% | 0.400% | 0.400% |
| Titanium Dioxide | 0% | 0% | 0% | 0.525% |
| Sodium Phosphate | 0% | 0% | 0% | 0.4% |
| Carboxymethylcellulose | 1.0% | 1.0% | 1.0% | 1.0% |
| Sodium | | | | |
| Flavor | 1.0% | 1.0% | 1.0% | 1.5% |
| WS5 Coolant | 0% | 0% | 0.2% | 0.15% |
| Sodium Lauryl Sulfate | 1.0% | 1.0% | 3.0% | 4.0% |
| 28% | | | | |
| Silica, Dental Type, | 10.0% | 15.0% | 15.0% | 10.0% |
| Zeodent 165 | | | | |
| Silica, Dental Type, | 5% | 5% | 5% | 5% |
| Zeodent 124 | | | | |
| Sorbitol Solution USP | 50% | 30% | 30% | 40% |
| Water Purified USP | 20.0% | 30.0% | 30.0% | 25.0% |
| Cocoamidopropyl | 3.0% | 3.0% | 0% | 0% |
| Betaine | | | | |
| Xanthan Gum | 1.0% | 1.0% | 1.0% | 1.5% |
| Cannabidiol (98%) | 0.5% | 0.1% | 0.25% | 0.5% |
| Stannous Chloride | 2.0% | 2.0% | 2.0% | 2.0% |
| Carbopol 946 | 0% | 0% | 0% | 0.3% |
| Ascorbic Acid | 0.4% | 0.4% | 0.4% | 0.4% |
| Vitamin E | 0% | 1.0% | 1.0% | 1.0% |
| Water | QS | QS | QS | QS |

The stability of Example A is measured using an HPLC analysis and the resulting data are shown in TABLE 2.

TABLE 2

| | Day 0 | Day 25 |
|---|---|---|
| Example A | Formulated CBD | Measured CBD |
| Value (mg/ml) | 5.0 | 3.8 |

*analyzed by HPLC

As can be seen in TABLE 2, the initial formulation of 5 mg/ml is stabilized at 3.8 mg/ml after 25 days in the toothpaste composition. Thus, the data in TABLE 2 shows that the cannabidiol formulated into Example A is highly stable over the course of 25 days from making. Without wishing to be bound by theory, it is believed that the Lewis acid and stannous chloride, helps to prevent the rapid oxidation and instability traditionally associated with cannabidiol formulated into compositions.

A non-fluoride based toothpaste having the formula of Example E as shown in TABLE 3 is made on a 10 Kg scale and then evaluated for CBD stability over the course of two months.

TABLE 3

| Ingredient | E |
|---|---|
| Sodium Saccharin | 0.65% |
| Sodium Gluconate | 2.0% |
| Sodium | 0.75% |
| Carboxymethylcellulose | |
| Peppermint Flavor | 1.5% |
| Sodium Lauryl Sulfate | 7.5% |
| (30% solution) | |
| Silica, Dental Type, | 5.0% |
| Zeodent 165 | |
| Silica, Dental Type, | 5.0% |
| Zeodent 124 | |
| Sorbitol Solution USP | 56% |
| Water Purified USP | 10% |
| Trisodium Phosphate | 1.1% |
| Xanthan Gum | 0.25% |
| Cannabidiol (98%) | 0.1% |
| Stannous Chloride | 2.0% |
| Ascorbic Acid (99%) | 0.4% |
| Butylated | 0.255 |
| Hydroxyanisole | |
| Glycerol | QS |

Exemplary deodorant and antiperspirant compositions F and G are made using a 10 Kg scale.

TABLE 4

| | Example | |
|---|---|---|
| Ingredient | F | G |
| Dipropylene glycol | 40% | — |
| Aluminum zirconium | — | 26% |
| trichlorohydrexglycinate | | |
| Pentylene glycol | 5% | — |
| Propylene glycol | 10% | — |
| 1,2-hexanediol | — | 5.0% |
| Glycerin | 15% | |
| Decamethylcyclopentasiloxane | — | 30% |
| (D5) | | |
| Dimethiconol | — | 10% |
| Tetrasodium EDTA | 0.1% | — |
| Sodium Stearate | 15% | — |
| Stearyl Alcohol | — | 15% |
| Sodium hydroxide | 0.05% | |
| Hydrogenated Castor Oil | — | 4.8% |
| PPG-3 Myristyl Ether | 2.0% | — |
| Stannous Chloride | 0.5% | — |
| Talc | — | 5% |
| Silica | — | 2% |
| Polyoxamine 1307 | 3.0% | — |
| CBD | 0.2% | 0.2% |
| Fragrance | 1.0% | 2.0% |
| Propylene glycol | QS | QS |

The stability of the CBD contained in Examples F and G is measured and evaluated over the course of two months. The resulting data are shown in TABLE 5. The data show that the oral care composition retains about 83% of the formulated CBD and the deodorant composition retained about 67% of the CBD. The deodorant was hot processed and this likely a degradation of the CBD, whereas the toothpaste was processed at room temperature.

TABLE 5

| | Day 0 | Day 60 | % CBD |
|---|---|---|---|
| | Formulated CBD | Measured CBD | Retention |
| Formula E, table 3 | 1 mg/g | 0.83 mg/g | 83% |
| Formula F, table 4 | 2 mg/g | 1.26 mg/g | 67% |

TABLE 6
Hair Care Products

| | A | B | C | D |
|---|---|---|---|---|
| selenomethionine | 0.4 | 0.2 | 0.1 | 0.8 |
| CBD | 0.1 | 0.25 | 0.5 | 1.0 |
| Ammonium Laureth Sulfate | 10 | 14 | 10 | 10 |
| Puresyn6(1-decenehomopolymer) | 0.4 | 0 | 0.4 | 0.4 |
| Trimethylolpropane Tricaprylate | 0.1 | 0 | 0.1 | 0.1 |
| Cocamide MEA | 0.8 | 0.8 | 0.8 | 0.8 |
| Citric Acid | 0.04 | 0 | 0.04 | 0.04 |
| Sodium Citrate Dihydrate | 0.4 | 0 | 0.4 | 0.4 |
| Disodium EDTA | 0.1 | 0 | 0.1 | 0.1 |
| Kathon | 0.0005 | 0 | 0.0005 | 0.0005 |
| DMDM Hydantoin | | 0.25 | | |
| Sodium Benzoate | 0.25 | 0 | 0.25 | 0.25 |
| Disodium EDTA | 0.1274 | 0 | 0.1274 | 0.1274 |
| CetylAlcohol | 0.9 | 0.6 | 0.9 | 0.9 |
| Stearyl Alcohol | 0 | 0.2 | 0 | 0 |
| EthyleneGlycolDistearate | 1.5 | 1.5 | 1.5 | 1.5 |
| Ammonium LaurylSulfate | 4.0 | 0 | 4.0 | 4.0 |
| Cocaminopropionicacid(2) | 2.0 | 0 | 2.0 | 2.0 |
| Lauraminopropionicacid(3) | 2.5 | 0 | 2.5 | 2.5 |
| SodiumLauriminodipropionate(4) | 0.85 | 0 | 0.85 | 0.85 |
| Polyquaternium-10 (KG30M) | 0.5 | 0 | 0.5 | 0.5 |
| GuarHydroxypropyltrimoniumChloride(1) | 0.5 | 0 | 0.5 | 0.5 |
| Polyquaternium-10(LR3OM) | 0.5 | 0.15 | 0.5 | 0.5 |
| Dow Corning 1664 300 nm/60M | 2.0 | 0 | 2.0 | 2.0 |
| emulsion | | | | |
| Dimethicone (Viscasil330M) | 2.35 | 1.0 | 2.35 | 2.35 |
| Perfume | 0.7 | 1.3 | 0.7 | 0.7 |
| Sodium Chloride 0-3 | 0-3 | 0 | 0-3 | 0-3 |
| Ammonium Xylene Sulfonate 0-3 | 0-3 | 0 | 0-3 | 0-3 |
| PEG150 1Pentaerythrityl Tetrastearate | 0.05 | 0 | 0.05 | 0.05 |
| (Crothix) | | | | |
| | 0 | 0 | 0 | 0 |
| Cocamidopropyl betaine | 0 | 2.7 | 0 | 0 |
| Mineral Oil | 0 | 0.3 | 0 | 0 |
| Cocamide MEA | 0 | 0.8 | 0 | 0 |
| Water | QS | QS | QS | QS |

(1)Guar having a molecular weight of about 400,000 and having a charge density of about 2.10 meq/g, available from Aqualon.
(2)Mackam151C(40% active), McIntyreGroupLtd.
(3)Mackam151L(40% active), McIntyreGroupLtd.
(4)Mackam160C-30(30% active), McIntyreGroupLtd.

TABLE 7
Pet Food and Pet Treat Products

| | A | B | C | D |
|---|---|---|---|---|
| selenomethionine | 2.5 | 1.0 | | |
| CBD | 0.5 | 0.2 | | |
| ExtractofAvocado* | 0.02 | 0 | | |
| Chicken, ChickenBy- | 44 | 0 | | |
| productMeal and | | | | |
| Fish Meal | | | | |
| Chicken Fat | 8.0 | 3.0 | | |
| BeetPulp | 2.0 | | | |
| Salts | 2.5 | | | |
| Vitamins** | 1.0 | 0.06 | | |
| Minerals** | 0.02 | 0.04 | | |
| Minors | 3.5 | 0 | | |
| Mannoheptulose | 0 | 0.14 | | |
| Spray-Dried Beef | 0 | 3.0 | | |
| Particles and Broth | | | | |
| Xanthan Gum | 0 | 0.5 | | |
| Flax Seed | 0 | 0.2 | | |
| Vegetables | 0 | 0.2 | | |
| Phosphoric Acid | 0 | 0.95 | | |
| Beef Flavor | 0 | 1.0 | | |
| Water | 0 | QS | | |
| Grains (Corn and | QS | | | |
| Sorghum) | | | | |

*Avocado may be substituted with other plant matter having enhanced mannoheptulose Content.
**Vitamins and Minerals include: VitaminE, beta-carotene and Vitamin A, Zinc Oxide, Ascorbic Acid, Manganese Sulfate, Copper Sulfate, Manganous Oxide, Calcium Pantothenate, Biotin, VitaminB12, VitaminB1, Niacin, VitaminB2, Vitamin B, Vitamin D, Folic Acid.

TABLE 8
Pet Chew Composition

| | A | B | |
|---|---|---|---|
| Wheat Protein Isolate | 15.8 | 17 | 0 |
| Soy Protein Isolate | 13.6 | 12 | 0 |
| Fibrous Protein | 0 | 0 | 30-50% |
| Gelling Protein (Gelatin 100 | 19.3 | 15 | 15-25% |
| Bloom) | | | |
| CBD | 0.1 | 0.8 | 0.005-10% |
| Delta Damascone | 0.05 | 0.1 | 0.001-5% |
| Ascorbic Acid | 0.2 | 0.4 | 0.001-5% |
| Sodium Caseinate | 8.9 | 8 | 0 |
| Glycerin | 15.8 | 17 | 15-25% |
| Hydrogenated Starch | 2.5 | 9 | 0-15% |
| Hydrolysate | | | |
| Water | 8.3 | 7 | 5-15% |
| Fat (Corn Oil or Fish Oil) | 1.4 | 3 | 1-10% |
| Bovine Colostrum | 0.01 | 5.0 | 0.005-20% |
| Flavor, Nutrients, Preservatives, | QS | QS | QS |
| Colorant | | | |

### Further Examples

A first exemplary composition for use in personal care compositions comprising:
i) A cannabinoid
ii) A Lewis acid salt
iii) A formula base capable of housing the cannabinoid and Lewis acid salt wherein the formula base may yield one or more of the following personal care compositions: oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats.

A second exemplary composition according to the first exemplary composition may contain a Michael Acceptor.

A third exemplary composition according to the either one of the first or second exemplary compositions may contain an antioxidant.

A fourth exemplary composition according to any one of the foregoing exemplary compositions may contain a flavor and/or fragrance.

A fifth exemplary composition according to any one of the foregoing exemplary compositions may be incorporated into one or more absorbent articles, such as disposable absorbent articles, having a lotion treatment composition applied thereon.

A sixth exemplary composition according to any one of the foregoing exemplary compositions may be incorporated into disposable absorbent articles which can be selected from one or more of the following: baby diapers or feminine hygiene articles, including incontinence devices and catamenial products, such as tampons, sanitary napkins, pantiliners, interlabial products, and the like.

I claim/We claim:

1. An oral care composition comprising in a single phase:
(a) a cannabinoid;
(b) a Lewis acid salt; and
(c) an orally-acceptable vehicle.

2. The oral care composition of claim 1, wherein the Lewis acid salt has formula I:
M′(Q)ᵣ
wherein:
(a) M′ is a metal or metalloid in its highest oxidation state;
(b) Q is a ligand selected from: hydride radicals, dialkyamido radicals, alkoxide radicals, aryloxide radicals, hydrocarbyl radicals, substituted hydrocarbyl radicals, halocarbyl radicals, substituted halocarbyl radicals, hydrocarbyl organometalloid radicals and halocaryl-substituted organometalloid radicals; and
(c) r is a number of Q ligands present in formula I.

3. The oral care composition of claim 2, wherein:
(a) M′ is selected from: boron, manganese, vanadium, iron, copper, stannous or selenium; and
(b) Q is selected from: mineral acids, chlorine, fluorine, sulfur, nitrogen, oxygen, phosphorous or amino acid;
wherein the amino acid is selected from: methionine, cysteine and combinations thereof.

4. The oral care composition of claim 1, wherein the orally-acceptable vehicle comprises a thickening agent comprising a polymer system selected from: a cross-linked polyvinylpyrrolidone, a gum, a cellulose derivative, a thickening silica and mixtures thereof.

5. The oral care composition of claim 1, further comprising at least one polyphosphate selected from: an alkali metal salt of a monophosphate, an alkali metal salt of a pyrophosphate, an alkali metal salt of a tripolyphosphate, hexametaphosphate and mixtures thereof.

6. The oral care composition of claim 4, wherein the alkali metal salt of a tripolyphosphate is selected from: tetrasodium pyrophosphate, sodium tripolyphosphate and mixtures thereof.

7. The oral care composition of claim 1, further comprising a source of fluoride ions and a source of stannous ions.

8. The oral care composition of claim 1, wherein the cannabinoid is selected from: a tetrahydrocannabinol (THC), an acid of tetrahydrocannabinol (THC), a cannabidiol (CBD), an acid of cannabidiol (CBD), a cannabigerol (CBG), an acid of cannabigerol (CBG) and mixtures thereof.

9. The oral care composition of claim 1, wherein the Lewis acid salt is selected from: stannous chloride, selenomethionine, selenocysteine, amines/polyamines and mixtures thereof.

10. The oral care composition of claim 1, wherein the oral care composition is in a product form selected from a: toothpaste, dentifrice, tooth gel, tooth powder, tablet, rinse, subgingival gel, foam, mousse, chewing gum, lipstick, sponge, floss, prophy paste, petrolatum gel, denture product and combinations thereof.

11. An oral care product comprising a first tube and a second tube wherein:
(a) the first tube contains a first composition comprising:
i. a first orally acceptable vehicle;
ii. a source of cannabinoid;
iii. a Lewis acid salt selected from: stannous chloride, selenomethionine, selenocysteine, amines/polyamines and mixtures thereof; and
iv. one or more Michael Acceptors and/or an antioxidant; and
(b) the second tube contains a second composition comprising:
i. fluoride salt;
ii. binder;
iii. humectant;
iv. abrasive; and
v. a second orally acceptable vehicle.

12. The oral care product of claim 11, wherein the cannabinoid is selected from: a tetrahydrocannabinol (THC), an acid of tetrahydrocannabinol (THC), a cannabidiol (CBD), an acid of cannabidiol (CBD), a cannabigerol (CBG), an acid of cannabigerol (CBG) and mixtures thereof.

13. The oral care product of claim 11, comprising a Michael acceptor selected from: delta damascene; dihydrojasmone; ascorbic acid [3-oxo-L-gulofuranolactone]; cis-jasmone[3-methyl-2-(2-pentenyl-2-cyclopentenone]; 2,5-dimethyl-4-hydroxy-3 (2H)-furanone; 5-ethyl-3-hydroxy-4-methyl-2(5H)-furanone; vanillin[4-hydroxy-3-methoxybenzaldehyde]; ethyl vanillin; anisaldehyde[4-methoxybenzaldehyde]; 3,4-methylenedioxybenzaldehyde; 3,4-dimethoxybenzaldehyde; 4-hydroxybenzaldehyde; 2-methoxybenzaldehyde; benzaldehyde; cinnamaldehyde[3-phenyl-2-propenal]; hexyl cinnamaldehyde; .alpha.-methyl cinnamaldehyde; ortho-methoxy cinnamaldehyde; citral; linalool; geraniol; eugenol; and mixtures thereof.

14. A personal care composition comprising:
(a) a formula vehicle;
(b) a cannabinoid;
(c) an emulsifier; and
(d) one or more antioxidants.

15. The personal care composition of claim 14, wherein the cannabinoid is selected from: a tetrahydrocannabinol (THC), an acid of tetrahydrocannabinol (THC), a cannabidiol (CBD), an acid of cannabidiol (CBD), a cannabigerol (CBG), an acid of cannabigerol (CBG) and mixtures thereof.

16. The personal care composition of claim 14, wherein the one or more antioxidants are selected from: a water-soluble antioxidant, an oil soluble antioxidant and mixtures thereof.

17. The personal care composition of claim 16 wherein the antioxidant is a water-soluble antioxidant selected from: glutathione, ascorbic acid, selenium, selenium derivatives, stannous chloride and mixtures thereof.

18. The personal care composition of claim 16 wherein the antioxidant is an oil soluble antioxidant selected from: butylated hydroxytoluene, butylated hydroxyaniline, carotenoids, tocopherol, tocopherol derivatives, stannous chloride and mixtures thereof.

19. A personal care composition comprising:
(a) a formula vehicle;
(b) a cannabinoid;
(c) an emulsifier;
(d) one or more antioxidants; and
(e) a dimethicone.

20. The personal care composition of claim 14, wherein the dimethicone is volatile at about 25 degrees Centigrade.
